(19) United States
(12) Patent Application Publication
(10) Pub. No.: US 2022/0389052 A1
(43) Pub. Date: Dec. 8, 2022

(54) GREEN AND BROAD-SPECTRUM PROTEIN CROSS-LINKING METHOD

(71) Applicant: LANZHOU UNIVERSITY, Lanzhou, Gansu (CN)

(72) Inventors: Jianxi XIAO, Lanzhou City, Gansu Province (CN); Jianrui Ma, Lanzhou City, Gansu Province (CN); Huxia He, Lanzhou City, Gansu Province (CN)

(21) Appl. No.: 17/782,649

(22) PCT Filed: Dec. 8, 2020
(86) PCT No.: PCT/CN2020/134395

§ 371 Date: Jun. 4, 2022

(30) Foreign Application Priority Data
Dec. 8, 2019 (CN) 201911246752.5

(51) Int. Cl. C07K 1/107; C07K 14/78

(52) U.S. Cl. CPC C07K 1/1075; C07K 14/78

(57) ABSTRACT

The present invention belongs to the technical field of medical biological materials, in particular to a green and broad-spectrum protein cross-linking method. In the method, a mixed solution containing protein and silver ions is irradiated by a visible light source with a compound wavelength to obtain cross-linked protein materials with uniform morphology. Compared with the traditional protein cross-linking method, the preparation method does not involve toxic chemical reagents and is environmentally friendly; the method can cross-link a variety of proteins in a broad spectrum; the preparation method can ensure the original activity of protein to the greatest extent; the preparation method also has the advantages of simple steps and easy operation; the cross-linked protein prepared by the method has good biological activity and antibacterial properties, and has great application prospect.

### TECHNICAL FIELD

The present invention belongs to the technical field of medical biological materials, in particular to a green and broad-spectrum protein cross-linking method.

### BACKGROUND TECHNOLOGY

In the field of tissue engineering, protein biomaterials can be used as the base material for tissue and organ substitutes—bricks, which have biological activity and biodegradability, and can repair, maintain and improve damaged tissues. They can be used for wound dressings, bionic skins, cardiovascular tissues (such as blood vessels, heart valves, myocardial patches, etc.), musculoskeletal tissues (such as cartilage, tendon, ligament, meniscus, bone, etc.), skin repair scaffolds and other fields of tissue engineering and regenerative tissue scaffolds^{[1]}.

However, protein and other bioactive materials currently used in protein biomaterials have poor mechanical properties, and they cannot provide sufficient structural support for protein biomaterials and achieve the mechanical properties and biodegradation rates required in biomedical applications, thus limiting their applications in biomedical materials and tissue engineering. For example, Chinese patent CN105936671A points out that unmodified collagen often has disadvantages such as poor thermal stability, rapid degradation rate, low mechanical strength and variability, which cannot meet the requirements for application in many cases; Chinese patent CN103333508A points out that uncross-linked collagen degrades quickly in the body and has a short retention time, which also limits its application in filling materials. Therefore, it is often necessary to cross-link the protein in one or more steps before applying the protein. Through the protein cross-linking technology, the thermal stability and mechanical strength of proteins can be improved, and the degradation rate of various bioactive proteins including collagen can be reduced, then the proteins can be widely used in many fields such as hemostatic materials, drug sustained-release carrier materials, tissue engineering scaffold materials, skin substitutes, bone graft materials, and corneal graft materials.

Protein cross-linking refers to the combination of two or more protein molecules through covalent bonds to improve the mechanical properties of protein materials, such as toughness, tensile resistance and impact resistance. At present, the commonly used cross-linking methods mainly include chemical cross-linking, enzyme cross-linking, high-temperature heat treatment cross-linking, freezing cross-linking, severe dehydration cross-linking and ultraviolet (UV) irradiation cross-linking, etc., but the above methods have the following problems: \textcircled{1} Chemical cross-linking method requires the addition of chemical cross-linking agents, among which the commonly used chemical cross-linking agents are glutaraldehyde (GA) or carbodiimide (EDC), which have certain toxicity and are not friendly to the environment. In addition, when chemical cross-linking agents are used in the process of protein cross-linking, the residual reagents have potential biotoxicity. For example, in Chinese patent CN101005865A, glutaraldehyde is used for cross-linking, but there is high concentration of glutaraldehyde residue in the product, which will cause strong biotoxicity and harm human health; \textcircled{2} High-temperature cross-linking method can change or even denature the functional structure of protein and lose the biological activity. Generally, it is only used for gel cross-linking, not for protein cross-linking. For example, Chinese patent CN109248337A discloses a preparation method of artificial dermal repair material, which needs to be cross-linked at 105° C. for 24 hours under the high temperature and vacuum conditions, but the structure and properties of protein are easily damaged at 105° C.; \textcircled{3} UV irradiation method generally requires the addition of light inducers, and the range of cross-linked proteins is limited; \textcircled{4} Severe dehydration method also requires high temperature treatment, which easily leads to the loss of protein activity; \textcircled{5} Enzyme cross-linking method is only applicable to proteins with specific structures and has poor broad spectrum. Chinese patent CN109908405A points out that some enzyme cross-linking agents also cause poor mechanical properties and easy swelling of the prepared bone scaffold materials due to their cross-linking properties; \textcircled{6} Freezing cross-linking method has poor cross-linking effect and generally needs to be combined with chemical cross-linking method, which is only suitable for the preparation of porous protein materials^{[2]}.

Therefore, it is very necessary to develop a simple, easy-to-control, safe, green, environmental-friendly, and broad-spectrum cross-linking method universal for proteins. The inventors unexpectedly found that cross-linked protein materials with uniform morphology and stable structure can be prepared by irradiating the mixed solution of protein and silver ions with a visible light source (380-780 nm). Compared with other methods, this method can be applied to the cross-linking of a variety of proteins with broad spectrum and good cross-linking effect, and the prepared cross-linked protein has good antibacterial properties without additional antibacterial agents required. In addition, this method has the advantages of mild conditions and green environmental protection, which can not only maintain the biological activity of proteins, but also significantly improve the mechanical properties of proteins. The prepared cross-linked protein can be used to prepare hemostatic materials, drug sustained-release carrier materials, tissue engineering scaffold materials, artificial skin, bionic teeth, bone repair materials and corneal graft materials with both biological activity and mechanical properties.

### REFERENCES

[1] Miranda-Nieves, David, Chaikof, Elliot L. Collagen and Elastin Biomaterials for the Fabrication of Engineered Living Tissues[J]. Acs Biomaterials Science & Engineering: acsbiomaterials 0.6b00250.
[2] Bi Binbin, Lin Qiaojia, Zheng Peitao, et al. Effects of Freezing Methods on the Structure and Adsorption Properties of Aldehyde-Crosslinked Soybean Protein Porous Materials [J]. Transactions of the Chinese Society of Agricultural Engineering, 2016, v. 32; No. 284(07): 317-322.

### CONTENTS OF THE INVENTION

In view of the above technical problems, the present invention aims to provide a protein cross-linking method, in which a compound wavelength light source or a single wavelength light source with a wavelength range of 380-780 nm is used to irradiate a mixed solution containing protein and Ag⁺.

Preferably, the concentration ratio of Ag⁺ to protein in the mixed solution is 1:1-100. Preferably, the concentration ratio of Ag⁺ to protein in the mixed solution is 1:10. Preferably, the irradiation temperature is 0-37° C.

Preferably, the irradiation time is 18 min-24 h.

Preferably, the irradiation time is 12 h.

Preferably, the proteins include collagen, bovine serum albumin, human serum albumin, egg white, casein, pepsin, and papain.

Preferably, the protein is collagen.

The other purpose of the present invention is to provide a method for preparing cross-linked protein, in which a compound wavelength light source or a single wavelength light source with a wavelength range of 380-780 nm is used to irradiate a mixed solution containing protein and Ag⁺, and then the cross-linked protein is obtained after reaction and centrifugation.

Preferably, the concentration ratio of Ag⁺ to protein in the mixed solution is 1:1-100. Preferably, the concentration ratio of Ag⁺ to protein in the mixed solution is 1:10. Preferably, the reaction temperature is 0-37° C.

Preferably, the reaction time is 18 min-24 h.

Preferably, the reaction time is 12 h.

Preferably, the proteins include collagen, bovine serum albumin, human serum albumin, egg white, casein, pepsin, and papain.

Preferably, the protein is collagen.

The present invention also aims to provide a cross-linked protein prepared by the above method.

Another purpose of the present invention is to provide a cross-linked protein applied in the preparation of hemostatic materials, drug sustained-release carrier materials, tissue engineering scaffold materials, artificial skin, bionic teeth, artificial blood vessels, bone repair materials, and corneal graft materials.

The beneficial effects of the present invention are as follows: \textcircled{1} The method provided by the present invention does not involve toxic chemical reagents and is environmentally friendly; \textcircled{2} The method of the present invention has good cross-linking effect on various proteins such as collagen, bovine serum albumin, human serum albumin, egg white, casein, pepsin, papain, etc., and is applicable to a broad spectrum; \textcircled{3}\textcircled{4} The method of the present invention can ensure the original activity of the protein to the greatest extent, and the prepared cross-linked protein has uniform morphology and stable properties; The cross-linked protein prepared by the method of the present invention has good antibacterial properties and does not need to add other antibacterial agents; \textcircled{5} The method of the present invention is simple and convenient, easy to operate and has a great application prospect; \textcircled{6} The cross-linked protein prepared by the present invention is safe and non-toxic, has good biological activity and mechanical properties, and can be widely used to prepare hemostatic materials, drug sustained-release carrier materials, tissue engineering scaffold materials, artificial skin, bionic teeth, bone repair materials and corneal graft materials.

### DESCRIPTION OF FIGURES

FIG. 1 Ultraviolet (UV) chromatographic, energy dispersive X-ray analysis (EDX), and X-ray photoelectron spectroscopy (XPS) images of cross-linked collagen, in which FIG. 1A (a-g in the figure respectively are the cross-linked collagen a-g prepared in Embodiment 1) is the UV detection image, and FIG. 1B is the EDX detection image of the cross-linked collagen a prepared in Embodiment 1, and FIG. 1C and FIG. 1D are the XPS detection images of the cross-linked collagen a prepared in Embodiment 1;

FIG. 2 Scanning electron microscope (SEM) image, transmission electron microscope (TEM) image, infrared chromatogram (IR) image and thermogravimetric analysis (TGA) image of cross-linked collagen, in which FIG. 2a is the SEM image, FIG. 2b is local amplification of FIG. 2a, FIG. 2c is the TEM image, FIG. 2d is local amplification of FIG. 2c, FIG. 2e is the IR detection image, and FIG. 2f is the TGA detection image;

FIG. 3 TEM images of cross-linked collagen prepared at different cross-linking times, in which the cross-linking times of FIG. 3a-f are 18 min, 30 min, 1 h, 3 h, 6 h, and 10 h, respectively;

FIG. 4 UV and TGA images of collagen prepared at different cross-linking times, in which FIG. 4a is the UV image and FIG. 4b is the TGA image;

FIG. 5 SEM and TEM images of cross-linked collagen prepared with different concentrations of collagen, in which FIG. 5a-d (protein concentrations are 1 mg/ml, 3 mg/ml, 5 mg/ml and 7 mg/ml, respectively) are SEM images, and FIG. 5e-h (protein concentrations are 1 mg/ml, 3 mg/ml, 5 mg/ml and 7 mg/ml, respectively) are TEM images;

FIG. 6 SEM and TEM images of cross-linked collagen prepared with different concentrations of AgNO₃, in which FIG. 6a-d (AgNO₃ concentrations are 0.1 mg/ml, 0.2 mg/ml, 0.5 mg/ml and 1 mg/ml, respectively) are SEM images, and FIG. 6e-h (AgNO₃ concentrations are 0.1 mg/ml, 0.2 mg/ml, 0.5 mg/ml and 1 mg/ml, respectively) are TEM images;

FIG. 7 SEM and TEM images of cross-linked collagen prepared at different pH values, in which FIG. 7a-d (pH 3, 5, 7, 9) are SEM images, and FIG. 7e-h (pH 3, 5, 7, 9) are TEM images;

FIG. 8 SEM and TEM images of other cross-linked proteins, in which FIG. 8A-F (the proteins are bovine serum albumin, casein, human serum albumin, pepsin, papain and egg white, respectively) are TEM images, and FIG. 8a-f (the proteins are bovine serum albumin, casein, human serum albumin, pepsin, papain and egg white, respectively) are SEM images.

### MODE OF CARRYING OUT THE INVENTION

In order to make the technical means, creative features, achievement goals and effects realized by the present invention easy to understand, the present invention is further described below in combination with the specific implementation mode. However, the protection scope of the present invention is not limited to the following embodiments. The methods used in one or more of the following embodiments are conventional unless otherwise specified; the materials, reagents, and other items used can be obtained commercially unless otherwise specified.

The proteins described in one or more of the following embodiments include collagen (CL), bovine serum albumin (BSA), casein, human serum albumin (HSA), pepsin, papain, and egg white.

The collagen (CL) described in one or more of the following embodiments is a biopolymer, which is the main component of connective tissue of animals and the most abundant and widely distributed functional protein in mammals. It consists of three polypeptide chains with a left-handed helical structure intertwined to form a right-handed helical structure.

The collagen (CL) described in one or more of the following embodiments may be collagen prepared from a variety of natural sources or by other means, such as natural collagen, recombinant collagen, and bionic collagen.

The Ag⁺ solution described in one or more of the following embodiments is AgNO₃ solution, and any Ag⁺ solution prepared with other soluble silver salts can be used for protein cross-linking.

The wavelength of visible light described in one or more of the following embodiments is 380-782 nm, and the light source is an incandescent lamp, but is not limited to the incandescent lamp. The light source can also be fluorescent lamp, flashlight, searchlight and any other light source that can emit visible light with a wavelength of 380-782 nm.

One or more of the following embodiments are carried out at room temperature, but it should be noted that the present invention realizes protein cross-linking without affecting protein stability and activity. Therefore, protein cross-linking can occur at temperatures that can maintain protein stability and activity (0-37° C.).

In one or more of the following embodiments where the reaction pH value is not specifically specified, the reaction pH value is 7.

The specific reaction parameters such as protein type, concentration, reaction time and lighting condition involved in protein cross-linking in all the following embodiments are shown in Table 1 below.

TABLE 1

| | | Protein | AgNO₃ | | | | |
|---|---|---|---|---|---|---|---|
| | | Concen- | Concen- | | | | |
| | Type of | tration | tration | | Reaction | | pH |
| S/N | Protein | (mg/ml) | (mg/ml) | Light | Time | | value |
| 1 | Collagen | 5 | 0.5 | Yes | 24 | h | 7 |
| 2 | Collagen | 5 | 0.5 | No | 24 | h | 7 |
| 3 | Collagen | 5 | 0.5 | Yes | 0 | h | 7 |
| 4 | Collagen | 5 | 0 | Yes | 24 | h | 7 |
| 5 | Collagen | 5 | 0 | Yes | 0 | h | 7 |
| 6 | Collagen | 0 | 0.5 | Yes | 24 | h | 7 |
| 7 | Collagen | 0 | 0.5 | Yes | 0 | h | 7 |
| 8 | Collagen | 1 | 0.5 | Yes | 12 | h | 7 |
| 9 | Collagen | 3 | 0.5 | Yes | 12 | h | 7 |
| 10 | Collagen | 5 | 0.5 | Yes | 12 | h | 7 |
| 11 | Collagen | 7 | 0.5 | Yes | 12 | h | 7 |
| 12 | Collagen | 5 | 0.1 | Yes | 12 | h | 7 |
| 13 | Collagen | 5 | 0.2 | Yes | 12 | h | 7 |
| 14 | Collagen | 5 | 1 | Yes | 12 | h | 7 |
| 15 | Collagen | 5 | 0.5 | Yes | 12 | h | 3 |
| 16 | Collagen | 5 | 0.5 | Yes | 12 | h | 5 |
| 17 | Collagen | 5 | 0.5 | Yes | 12 | h | 7 |
| 18 | Collagen | 5 | 0.5 | Yes | 12 | h | 9 |
| 19 | Collagen | 5 | 0.5 | Yes | 18 | min | 7 |
| 20 | Collagen | 5 | 0.5 | Yes | 30 | min | 7 |
| 21 | Collagen | 5 | 0.5 | Yes | 1 | h | 7 |
| 22 | Collagen | 5 | 0.5 | Yes | 3 | h | 7 |
| 23 | Collagen | 5 | 0.5 | Yes | 6 | h | 7 |
| 24 | Collagen | 5 | 0.5 | Yes | 10 | h | 7 |
| 25 | Human | 5 | 0.5 | Yes | 12 | h | 7 |
| | serum | | | | | | |
| | albumin | | | | | | |
| | (HSA) | | | | | | |
| 26 | Bovine | 5 | 0.5 | Yes | 12 | h | 7 |
| | serum | | | | | | |
| | albumin | | | | | | |
| | (BSA) | | | | | | |
| 27 | Egg white | 5 | 0.5 | Yes | 12 | h | 7 |
| 28 | Pepsin | 5 | 0.5 | Yes | 12 | h | 7 |
| 29 | Papain | 5 | 0.5 | Yes | 12 | h | 7 |
| 30 | Casein | 5 | 0.5 | Yes | 12 | h | 7 |

The cross-linked protein described in one or more of the following embodiments refers to the product formed after cross-linking of protein and Ag⁺ under visible light irradiation.

The cross-linked proteins prepared in one or more of the following embodiments have good mechanical properties and can be used to prepare hemostatic materials, drug sustained-release carrier materials, tissue engineering scaffold materials, artificial skin, bionic teeth, artificial blood vessels, bone repair materials, corneal graft materials and other medical instruments.

The power of the visible light source described in one or more of the following embodiments is 48 W, and the irradiation distance is 20 cm. However, the power and irradiation distance of the visible light source in the embodiments are not the only choices. The power and irradiation distance of the visible light source can be adjusted according to different experimental operations.

UV described in one or more of the following embodiments is ultraviolet spectrum, EDX is energy dispersive X-ray analysis, XPS is X-ray photoelectron spectroscopy, SEM is scanning electron microscope, TEM is transmission electron microscope, IR is infrared chromatography, and TGA is thermogravimetric analysis.

The UV detection described in one or more of the following embodiments is as follows: take 500 ul of the prepared cross-linked protein sample, dilute it to 3 ml, and then perform UV detection; the EDX detection described is as follows: take 500 ul of the prepared cross-linked protein sample, dilute it to 3 ml, and then perform EDX detection; the XPS detection described is as follows: take an appropriate amount of cross-linked protein sample and freeze-dry it, and then perform XPS detection; the SEM detection described is as follows: take an appropriate amount of cross-linked protein reaction solution, drop it on the silicon wafer and spray gold, and then perform SEM detection; the TEM detection described is as follows: take an appropriate amount of cross-linked protein reaction solution, drop it on the copper grid to dry, and then perform TEM detection; the IR detection described is as follows: take an appropriate amount of cross-linked protein sample and freeze-dry it, and then perform IR detection; the TGA detection described is as follows: take an appropriate amount of cross-linked protein sample and freeze-dry it, and then perform TGA detection.

### Embodiment 1

### 1.1 Preparation of Cross-Linked Collagen

Configuration of collagen solution: dissolve 10 mg collagen solid in 1 ml water, then configure it into a collagen solution with a concentration of 10 mg/ml, and dilute it with water before use.

Configuration of AgNO₃ solution: dissolve 100 mg AgNO₃ solid in 1 ml water, then configure it into an AgNO₃ solution with a concentration of 100 mg/ml, and dilute it with water before use.

### Preparation of Cross-Linked Collagen:

a. At room temperature, the collagen solution was added to the 12-well plate, and the visible light source with a power of 48 W was used to irradiate the collagen solution at a distance of 20 cm. The solution was stirred quickly and AgNO₃ solution was added slowly to obtain the final solution containing 5 mg/ml collagen and 0.5 mg/ml AgNO₃. The reaction solution a was prepared after reaction for 24 hours;
b. At room temperature, the collagen solution was added to the 12-well plate, the solution was stirred quickly and AgNO₃ solution was added slowly to obtain the final solution containing 5 mg/ml collagen and 0.5 mg/ml AgNO₃. The reaction solution b was prepared after reaction for 24 hours;
c. At room temperature, the collagen solution was added to the 12-well plate, and the visible light source with a power of 48 W was used to irradiate the collagen solution at a distance of 20 cm. The solution was stirred quickly and AgNO₃ solution was added slowly to obtain the final solution containing 5 mg/ml collagen and 0.5 mg/ml AgNO₃, and then the reaction solution c was prepared;
d. At room temperature, 5 mg/ml collagen solution was added to the 12-well plate, and the visible light source with a power of 48 W was used to irradiate the collagen solution at a distance of 20 cm for 24 hours, then the reaction solution d was prepared;
e. At room temperature, 5 mg/ml collagen solution was added to the 12-well plate, and then the reaction solution e was prepared;
f. At room temperature, 0.5 mg/ml AgNO₃ solution was added to the 12-well plate, and the visible light source with a power of 48 W was used to irradiate the AgNO₃ solution at a distance of 20 cm for 24 hours, then the reaction solution f was prepared;
g. At room temperature, 0.5 mg/ml AgNO₃ solution was added to the 12-well plate, and then the reaction solution g was prepared.

Purification of cross-linked protein: The above reaction solutions a-g were centrifuged at 9500 rpm, the supernatant was discarded to retain the precipitate, and the cross-linked collagens a-g were obtained after washing and centrifugation with deionized water for 3-5 times and then drying at room temperature.

### 1.2 Structural Detection of Cross-Linked Collagen

The cross-linked collagens a-g were detected by UV respectively; the cross-linked collagen a was detected by EDX, XPS, SEM, TEM, IR and TGA.

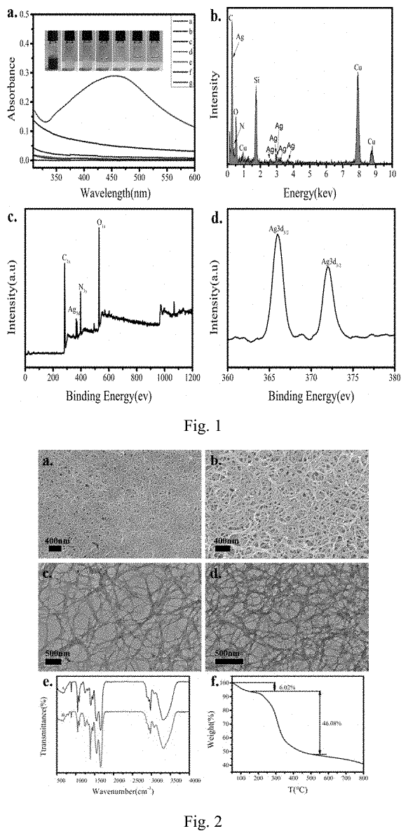

The UV detection results are shown in FIG. 1A (a-g in the figure are the cross-linked collagens a-g prepared above respectively), only when collagen and Ag⁺ exist at the same time, and under the visible light condition, the reaction system has an obvious absorption peak at the wavelength of about 450 nm, indicating the formation of silver nanoparticles; the EDX detection result is shown in FIG. 1B, the XPS detection result is shown in FIG. 1C and FIG. 1D, and the EDX and XPS detection peaks further indicate the generation of silver nanoparticles under such conditions; the SEM, TEM, IR and TGA detection results of the cross-linked collagen a prepared above are shown in FIG. 2, in which FIG. 2a is the SEM image, FIG. 2b is the local amplification of FIG. 2a, FIG. 2c is the TEM image, FIG. 2d is the local amplification of FIG. 2c, FIG. 2e is the IR detection image, and FIG. 2f is the TGA detection image; the SEM and TEM detection show that collagen fibers are formed in the reaction solution; the IR detection result shows that the tertiary structure of collagen is still intact; the TGA detection result shows that the collagen in the reaction system accounts for nearly half of the total amount of the system. The above experimental results show that only when the protein solution, silver ion solution and visible light condition exist at the same time, the protein is cross-linked, and the structure of the prepared cross-linked protein is intact.

### Embodiment 2

### 2.1 Preparation of Cross-Linked Collagen

The collagen solutions and AgNO₃ solutions were configured as shown in 1.1 above.

### Preparation of Cross-Linked Collagen:

At room temperature, the collagen solution was added to the 12-well plate, and the visible light source with a power of 48 W was used to irradiate the collagen solution at a distance of 20 cm. The solution was stirred quickly and AgNO₃ solution was added slowly to obtain the final solution containing 5 mg/ml collagen and 0.5 mg/ml AgNO₃. The reaction solutions were prepared by reaction for 18 min, 30 min, 60 min, 3 h, 6 h and 10 h, respectively.

Purification of cross-linked protein: The above reaction solutions were centrifuged at 9500 rpm, the supernatant was discarded to retain the precipitate, and the cross-linked collagens with different cross-linking times were obtained after washing and centrifugation with deionized water for 3-5 times and then drying at room temperature.

### 2.2 Structural Detection of Cross-Linked Collagen

The above-mentioned cross-linked collagens or the reaction solutions were detected by UV, TEM and TGA respectively.

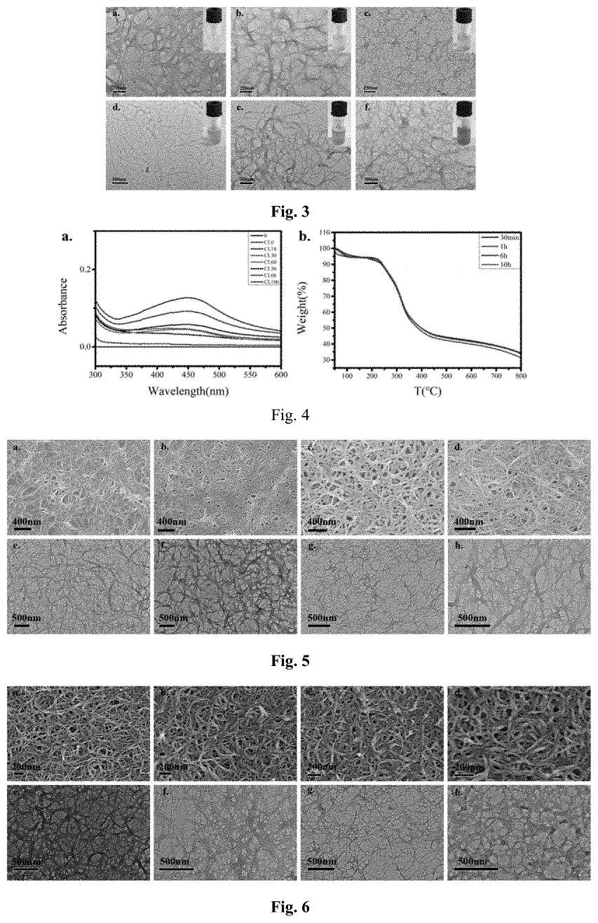

The TEM detection results are shown in FIG. 3, and collagen fibers with uniform morphology are generated in the reaction solutions with different cross-linking times; the UV detection results are shown in FIG. 4a, and with the extension of the cross-linking time, the absorption peak of the reaction system at the wavelength of about 450 nm increases significantly, indicating that the formation of silver nanoparticles increases gradually with the extension of cross-linking time; the TGA detection results are shown in FIG. 4b, and the thermogravimetric trend lines do not change, and the collagen in the reaction systems with different cross-linking times accounts for nearly half of the total amount of the systems, indicating that the content of collagen fibers in the system increases gradually with the increase of silver nanoparticles. The above results indicate that the cross-linked collagen can be formed when the cross-linking time is more than 18 min, and the content of collagen fibers in the reaction system increases with the extension of cross-linking time.

### Embodiment 3

### 3.1 Preparation of Cross-Linked Collagen

The collagen solutions and AgNO₃ solutions were configured as shown in 1.1 above.

### Preparation of Cross-Linked Collagen:

At room temperature, the collagen solution was added to the 12-well plate, and the visible light source with a power of 48 W was used to irradiate the collagen solution at a distance of 20 cm. The solution was stirred quickly and AgNO₃ solution was added slowly to obtain the final solutions containing collagen with different concentrations (1 mg/ml, 3 mg/ml, 5 mg/ml, 7 mg/ml) and 0.5 mg/ml AgNO₃. The reaction solutions were prepared after reaction for 12 hours.

Purification of cross-linked protein: The above reaction solutions were centrifuged at 9500 rpm, the supernatant was discarded to retain the precipitate, and the cross-linked collagens with different collagen concentrations were obtained after washing and centrifugation with deionized water for 3-5 times and then drying at room temperature.

### 3.2 Structural Detection of Cross-Linked Collagen

The above-mentioned cross-linked collagens or the reaction solutions were detected by TEM and SEM respectively.

The results are shown in FIG. 5, in which FIG. 5a-d are the SEM images of different collagen concentrations of 1 mg/ml, 3 mg/ml, 5 mg/ml and 7 mg/ml respectively, and FIG. 5e-h are the TEM images of different collagen concentrations of 1 mg/ml, 3 mg/ml, 5 mg/ml and 7 mg/ml respectively. The results show that collagen fibers with uniform morphology are generated in the reaction solutions with different collagen concentrations, indicating that the collagen concentration has little effect on collagen cross-linking, and collagen fibers with uniform morphology can be formed at any concentration.

### Embodiment 4

### 4.1 Preparation of Cross-Linked Collagen

The collagen solutions and AgNO₃ solutions were configured as shown in 1.1 above.

### Preparation of Cross-Linked Collagen:

At room temperature, the collagen solution was added to the 12-well plate, and the visible light source with a power of 48 W was used to irradiate the collagen solution at a distance of 20 cm. The solution was stirred quickly and AgNO₃ solution was added slowly to obtain the final solutions containing 5 mg/ml collagen and AgNO₃ with different concentrations (0.1 mg/ml, 0.2 mg/ml, 0.5 mg/ml and 1.0 mg/ml). The reaction solutions were prepared after 12 hours of reaction respectively.

Purification of cross-linked protein: The above reaction solutions were centrifuged at 9500 rpm, the supernatant was discarded to retain the precipitate, and the cross-linked collagens with different AgNO₃ concentrations were obtained after washing and centrifugation with deionized water for 3-5 times and then drying at room temperature.

### 4.2 Structural Detection of Cross-Linked Collagen

The above-mentioned cross-linked collagens or the reaction solutions were detected by TEM and SEM respectively.

The results are shown in FIG. 6, in which FIG. 6a-d are the SEM images of different AgNO₃ concentrations of 0.1 mg/ml, 0.2 mg/ml, 0.5 mg/ml and 1 mg/ml respectively, and FIG. 6e-h are the TEM images of different AgNO₃ concentrations of 0.1 mg/ml, 0.2 mg/ml, 0.5 mg/ml, 1 mg/ml respectively. The results show that collagen fibers with uniform morphology are generated in the reaction solutions with different AgNO₃ concentrations, indicating that AgNO₃ concentration has little effect on collagen cross-linking, and collagen fibers with uniform morphology can be formed at any concentration.

### Embodiment 5

### 5.1 Preparation of Cross-Linked Collagen

The collagen solutions and AgNO₃ solutions were configured as shown in 1.1 above.

### Preparation of Cross-Linked Collagen:

The pH value of the collagen solution was adjusted to 3, 5, 7 and 9 respectively, and at room temperature, the collagen solutions with different pH values were added to the 12-well plate, then the visible light source with a power of 48 W was used to irradiate the collagen solution at a distance of 20 cm. The solution was stirred quickly and AgNO₃ solution was added slowly to obtain the final solution containing 5 mg/ml collagen and 0.5 mg/ml AgNO₃. The reaction solutions were prepared after reaction for 12 hours respectively.

Purification of cross-linked protein: The above reaction solutions were centrifuged at 9500 rpm, the precipitate was retained, and the cross-linked collagens with different pH reactions were obtained after washing and centrifugation with deionized water for 3-5 times and then drying at room temperature.

### 5.2 Structural Detection of Cross-Linked Collagen

The above-mentioned cross-linked collagens or the reaction solutions were detected by TEM and SEM respectively.

The detection results are shown in FIG. 7, in which FIG. 7a-d are the SEM images at pH 3, 5, 7, and 9 respectively, and FIG. 7e-h are the TEM images at pH 3, 5, 7, and 9 respectively. The results show that collagen fibers with uniform morphology are generated in the reaction solutions with different pH values, indicating that cross-linked collagen can be formed in all reaction systems with different pH values.

### Embodiment 6

### 6.1 Preparation of Cross-Linked Protein

The protein solutions and AgNO₃ solutions were configured as shown in 1.1 above, where the proteins are BSA, Casein, HSA, Pepsin, Papain and Egg White respectively.

### Preparation of Cross-Linked Protein:

At room temperature, the above protein solutions were added to the 12-well plate, and the visible light source with a power of 48 W was used to irradiate the protein solution at a distance of 20 cm. The solution was stirred quickly and AgNO₃ solution was added slowly to obtain the final solution containing 5 mg/ml protein and 0.5 mg/ml AgNO₃. The reaction solution was prepared after reaction for 12 hours respectively.

Purification of cross-linked protein: The above reaction solutions were centrifuged at 9500 rpm, the supernatant was discarded to retain the precipitate, and the cross-linked proteins with different protein types were obtained after washing and centrifugation with deionized water for 3-5 times and then drying at room temperature.

### 6.2 Structural Detection of Cross-Linked Protein

The above-mentioned cross-linked proteins or the reaction solutions were detected by TEM and SEM respectively.

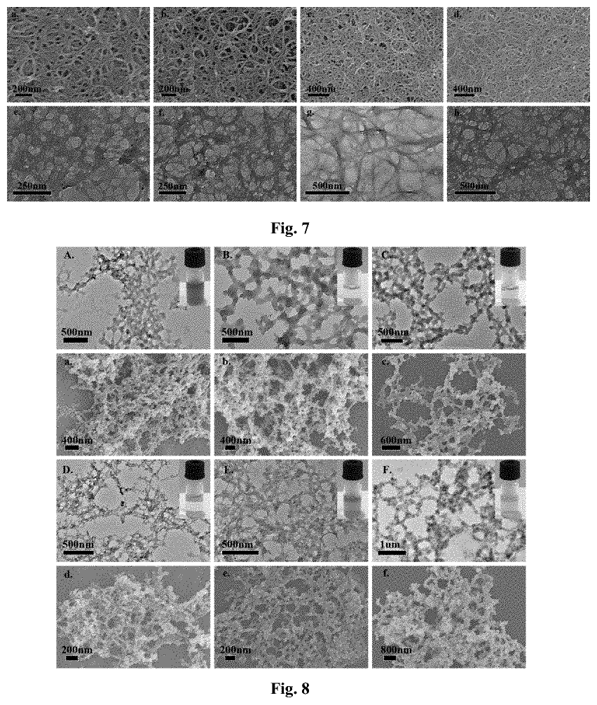

The detection results are shown in FIG. 8, in which FIG. 8A-F are the TEM images of BSA, casein, HSA, pepsin, papain and egg white respectively, and FIG. 8a-f are the SEM images of BSA, casein, HSA, pepsin, papain and egg white respectively. The results show that the fibers with uniform morphology are generated in the reaction systems of different proteins, indicating that the method is also suitable for the cross-linking of other proteins and has broad spectrum.

In summary, the present invention obtains a cross-linked protein by cross-linking the protein solution and the silver ion solution under visible light irradiation. The method is implemented under mild conditions without additional cross-linking agent required, and it is safe and non-toxic. The prepared cross-linked protein has good biological activity and antibacterial properties. Compared with the cross-linked protein prepared by other cross-linking methods, it is more suitable for the preparation of hemostatic materials, drug sustained-release carrier materials, tissue engineering scaffold materials, artificial skin, bionic teeth, artificial blood vessels, bone repair materials and corneal graft materials.

The above descriptions are only the details of individual exemplary implementation cases of the present invention. For the technical personnel in the field, the present invention can be modified and changed in the actual application process according to the specific preparation conditions, which is not intended to limit the present invention. Anything within the spirit and principle of the present invention shall be included in the protection scope of the present invention.

1. A protein cross-linking method is characterized in that the method comprises the following steps: a compound wavelength light source or a single wavelength light source with a wavelength range of 380-780 nm is used to irradiate a mixed solution containing protein and Ag⁺.

2. A method for preparing cross-linked protein is characterized in that the method comprises the following steps: a compound wavelength light source or a single wavelength light source with a wavelength range of 380-780 nm is used to irradiate a mixed solution containing protein and Ag⁺, and then the cross-linked protein is obtained after reaction and centrifugation.

3. The method, as stated in claim 2, is characterized in that the concentration ratio of Ag⁺ to protein in the mixed solution is 1:1-100.

4. The method, as stated in claim 3, is characterized in that the concentration ratio of Ag⁺ to protein in the mixed solution is 1:10.

5. The method, as stated in claim 2, is characterized in that the reaction temperature is 0-37° C.

6. The method, as stated in claim 2, is characterized in that the reaction time is 18 min-24 h.

7. The method, as stated in claim 6, is characterized in that the reaction time is 12 h.

8. The method, as stated in claim 2, is characterized in that the proteins include collagen, bovine serum albumin, human serum albumin, egg white, casein, pepsin, and papain.

9. The method, as stated in claim 8, is characterized in that the protein is collagen.

10. The cross-linked protein prepared by any of the methods stated in claim 9.

11. The application of cross-linked protein, as stated in claim 10, in the preparation of hemostatic materials, drug sustained-release carrier materials, tissue engineering scaffold materials, artificial skin, bionic teeth, artificial blood vessels, bone repair materials and corneal graft materials.
